(19) United States
(12) Patent Application Publication
(10) Pub. No.: US 2009/0198285 A1
(43) Pub. Date: Aug. 6, 2009

(54) SUBCONDYLAR FRACTURE FIXATION PLATE SYSTEM FOR TUBULAR BONES OF THE HAND

(76) Inventor: Raymond Raven, III, Glendale, CA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 12/367,291

(22) Filed: Feb. 6, 2009

Related U.S. Application Data
(60) Provisional application No. 61/026,725, filed on Feb. 6, 2008.

(51) Int. Cl. A61B 17/80

(52) U.S. Cl. 606/286; 606/280

(57) ABSTRACT

A medical implant plate including at least two sets of apertures through an elongated shaft body to individually accommodate and orient set screws or pegs at various angles that may be selected depending on application, i.e., the apertures don't have fixed angled, but allow for a range of angles. Once the screw has been locked, the device becomes a fixed angled device. The screws or pegs laterally spaced relative to each other to resist torsion and to secure the plate against dislodgement. The body also includes a flared-end portion that accommodates and extends partially onto a metaphysis of a tubular bone of the hand while maintaining a low-profile to avoid soft-tissue irritation. The flared-end portion that extends on to the metaphysis does NOT have holes for screws but serves as a buttress. Screws that extend into the metaphysis come from the three holes located along the slightly angled portion of the plate.

### CROSS-REFERENCE TO RELATED APPLICATION

This application for patent claims the benefit of priority from U.S. Provisional Patent Application Ser. No. 61/026,725 filed Feb. 6, 2008, the disclosure of which is incorporated herein by reference in its entirety.

### BACKGROUND OF THE INVENTION

1. Field of the Invention

The present general inventive concept relates generally to a fracture fixation plate system, and more particularly, to a fracture fixation plate system designed to stabilize a fracture of a tubular bone of the hand.

2. Description of the Related Art

Plates having fixed-angled locking features are known for use with distal radius (wrist) fractures, proximal humerus (shoulder) fractures and some hand fractures. These plates have revolutionized the treatment of fractures that have occurred in comminuted and/or osteoporotic bone, providing rigid and stable internal fixation and allowing for early range of motion.

Conventional wrist and shoulder plates have a raised head that extends onto the metaphysis of a bone to permit locking screws to extend into the metaphysis at a fixed-angle. These wrist and shoulder plates cover an increased area of the bone and provide increased support to the screws extending into the metaphysis.

Although conventional wrist and shoulder plates have a relatively high and bulky profile that is likely to protrude from the bone of the metaphysis, such protrusion is not a problem because muscles and other soft-tissue overlying the shoulder and wrist acts as a buffer between the plate and soft tissue, which significantly decreases the likelihood of tendon and other soft-tissue irritation.

Because the hand does not have the similar muscle mass to act as a buffer similar to the shoulder and forearm muscles, the body of conventional hand plates are designed to be much lower in profile. However, the low profile plating systems available must extend on to the metaphysis of tubular bones of the hand when used to stabilize fractures that extend into the metaphysis or into the nearby articular surface.

Following the fixation of such fractures using conventional hand plating systems, there may be cases in which the plate is prominent, causing tendon and soft-tissue irritation and pain. It is not uncommon, in such cases, for there to be a need for subsequent hardware removal once the fracture has healed, necessitating another surgery. Consequently, conventional hand plates are less than optimal for use in the treatment of certain types of hand fractures.

Accordingly, it would be beneficial to provide a fracture fixation plate system to stabilize a fracture of a tubular bone in a region having generally low muscle mass, such as the hand, that extends onto the metaphysis of the bone, but is not prominent, avoids soft-tissue irritation, and may be employed via minimally invasive means.

### SUMMARY OF THE INVENTION

The present general inventive concept provides a fixed-angled fracture fixation device to treat tubular hand bone fractures that extends onto the metaphysis or articular surface of the tubular hand bone.

The present general inventive concept also provides a fixed-angled fracture fixation device for rigid osteosynthesis.

The present general inventive concept also provides a fixed-angled fracture fixation device having a body that does not, itself, extend onto the metaphysis but has locking holes that receive fixed angled screws such that the screws are directed and extend into the metaphysis to facilitate fixation of those tubular hand bone fractures that extend into the metaphysis or articular surface.

The present general inventive concept also provides a fixed-angled fracture fixation device having a low profile that remains proximal or distal to a metaphysis of a tubular hand bone, thereby avoiding prominence and soft-tissue and/or tendon irritation.

The present general inventive concept also provides a fixed-angled fracture fixation device having a low profile that is less likely to require removal due to skin irritation and/or tendon ruptures.

The present general inventive concept also provides a fracture fixation plate that is designed to engage a tubular hand bone, and is not rough to touch.

The present general inventive concept also provides a fracture fixation plate that is subcondylar such that the fracture fixation plate is situated just below or above the condyle or metaphysis of a tubular hand bone.

The present general inventive concept also provides a fracture fixation plate that primarily resides on a diaphysis of a tubular hand bone.

The present general inventive concept also provides a fracture fixation plate for a metacarpal or phalanx.

The present general inventive concept also provides a fracture fixation plate having a fixed-angled locking feature.

The present general inventive concept also provides a fracture fixation plate having screws that extend into a metaphysis central portion of a tubular hand bone.

The present general inventive concept also provides a fracture fixation plate that may be placed in a minimally invasive fashion, e.g., requires only a small incision.

The present general inventive concept also provides a fracture fixation plate that is substantially rigid and manufactured from stainless steel, titanium alloy, or the like.

The present general inventive concept also provides a fracture fixation plate that provides proper alignment and stabilization of a tubular hand bone fracture.

The present general inventive concept also provides at least two fracture fixation plates of different sizes and/or shapes that cooperate to provide a fracture-fixation system for providing proper alignment and stabilization of tubular hand bone fractures.

The present general inventive concept also provides such an apparatus that is easy to use, comparatively simple to manufacture, and especially well adapted for the intended usage thereof.

Additional aspects and utilities of the present general inventive concept will be set forth in part in the description that follows and, in part, will be obvious from the description, or may be learned by practice of the general inventive concept.

The foregoing and/or other aspects and utilities of the present general inventive concept may be achieved by providing.

The foregoing and/or other aspects and utilities of the present general inventive concept may also be achieved by providing a tubular hand bone fracture fixation apparatus for use with a plurality of screws, the apparatus including an elongated body defining a longitudinal axis, the elongated body having a first portion and a second portion with an upper surface and a bone-engagement lower surface, a first set of apertures through the first portion to orient a number of the plurality of fixation screws along two or more axes, a second set of apertures through the second portion to orient a number of the plurality of fixation screws along a common axis, and an oblong aperture located within the second set of apertures to orient a set screw, wherein the first portion includes a support-enhancement element.

The apparatus may include a plurality of peaks along the upper surface between the second set of apertures.

A length of the plurality of peaks may successively increase from the second portion to the first portion.

The apparatus may include sidewalls of the elongated body extending along the longitudinal axis, wherein the sidewalls extend substantially parallel to each other along the second portion of the elongated body, and the sidewalls diverge along the first portion of the elongated body.

The apparatus may include a plurality of inward portions of the sidewalls that narrow the elongated body, and a plurality of outward portions of the sidewalls that widen the elongated body.

Each of the plurality of inward portions may be adjacent to a non-aperture area of the elongated body, and each of the plurality of outward portions may be adjacent to one of the second set of apertures.

A length of the plurality of outward portions may successively increase from the second portion to the first portion.

The first set of apertures may be conical tapered inward and angled with respect to a central axis of each of the apertures.

The support-enhancement element may have an interior with an upper wall and three walls that depend from the upper wall.

The support-enhancement element may be formed by parallel side surfaces of the elongated body that diverge outward from each other.

The support-enhancement element may be a buttress, a buttress cavity, or a buttress concavity.

The bone-engagement lower surface may flare upward and/or outward at the support-enhancement element to form a concavity that may extend onto a metaphyse of a tubular bone.

The screws may include a fixation screw and/or a set screw.

The upper surface and the bone-engagement lower surface of the elongated body may extend substantially parallel to each other along the second portion, and/or the upper surface may extend at an upward angle of approximately 13 degrees from the bone-engagement lower surface along the first portion.

The apparatus may include an elongated aperture extending axially in a direction substantially aligned with the longitudinal axis.

The elongated body may taper in thickness from the second portion to the first portion and may be thinnest proximate to an edge of the support-enhancement element.

Each of the first set of apertures and/or the second set of apertures may be conically tapered inward toward the bone-engagement lower surface of the elongated body.

The apparatus may be a metacarpal plate and/or phalanx plate and may be reversible along a metacarpal and/or phalanx.

The first set of apertures may include a lower first aperture that may be on the common axis of the second set of apertures, and/or upper second and third apertures that may be opposite to each other on either side of the first aperture on another axis that is perpendicular to the common axis.

The first set of apertures may orient one or more screws at an angle between 10 and 45 degrees relative to the planar top surface and along a z-axis thereof.

The elongated body may have a thickness of 1.6 mm, 2.0 mm, or 2.4 mm.

If only one of a first end or second end is a tapered surface, then a surface opposite to the tapered surface may be a planar surface along the entire length of the plate.

A first end surface may taper in thickness along one or both of the upper surface and the bone-engagement lower surface.

One or more of the second set of apertures may have a first diameter and a second diameter, the second diameter being different than the first diameter.

The second set of apertures may orient one or more of the plurality of fixation screws at an angle between 10 and 45 degrees relative to the planar top surface.

The screws are headless, locking, not locking, cannulated, not cannulated, or have threaded heads.

The screws may have a core diameter of 2.0 mm or 2.4 mm.

The first set of apertures and the second set of apertures may be predrilled locking holes to prevent inadvertent removal or toggling of the screws.

The apparatus may mount on a bone on a prominent area of the body such as a tubular hand bone such as a metacarpal and/or a phalanx.

### BRIEF DESCRIPTION OF THE DRAWINGS

These and/or other aspects and utilities of the present general inventive concept will become apparent and more readily appreciated from the following description of the embodiments, taken in conjunction with the accompanying drawings of which:

FIG. 1 is a top plan view of a fracture fixation plate system of the present general inventive concept illustrating a plurality of plates each resident on a tubular hand bone.

FIG. 2 is a side elevation view of a fracture fixation plate system of the present general inventive concept illustrating a plurality of fracture fixation plates each having a longitudinal planar surface, the fracture fixation plates attached to a metacarpal bone and a phalanx, each fracture fixation plate attached via a plurality of locking screws that extend into the hand bone at an angle that is perpendicular to the plate planar surface and three locking screws on the angled portion of the plate that is at an angle of approximately 12 degrees to the plate planar surface, the screw being fixed at angles of approximately 10-45 degrees to the plate planar surface.

FIG. 3 is a top plan view of a large fracture fixation plate of the present general inventive concept illustrating tapered or conical apertures and an oblong positioning hole that is non-locking.

FIG. 4 is a side elevation view of the large fracture fixation plate of the present general inventive concept illustrating a raised first end.

FIG. 5 is a bottom plan view of the large fracture fixation plate of the present general inventive concept.

FIG. 6 is a front elevation view of the large fracture fixation plate of the present general inventive concept illustrating the raised first end.

FIG. 7 is a rear elevation view of the large fracture fixation plate of the present general inventive concept illustrating a body with the first end rising above the body in the background.

FIG. 8 is a top plan view of a small fracture fixation plate of the present general inventive concept illustrating tapered or conical apertures.

FIG. 9 is a side elevation view of the small fracture fixation plate of the present general inventive concept illustrating a raised first end.

FIG. 10 is a bottom plan view of the small fracture fixation plate of the present general inventive concept.

FIG. 11 is a front elevation view of the small fracture fixation plate of the present general inventive concept illustrating the raised first end.

FIG. 12 is a rear elevation view of the small fracture fixation plate of the present general inventive concept illustrating a body with the first end rising above the body in the background.

### DETAILED DESCRIPTION OF THE PREFERRED EMBODIMENTS

Reference will now be made in detail to the embodiments of the present general inventive concept, examples of which are illustrated in the accompanying drawings, wherein like reference numerals refer to the like elements throughout. The embodiments are described below in order to explain the present general inventive concept be referring to the figures.

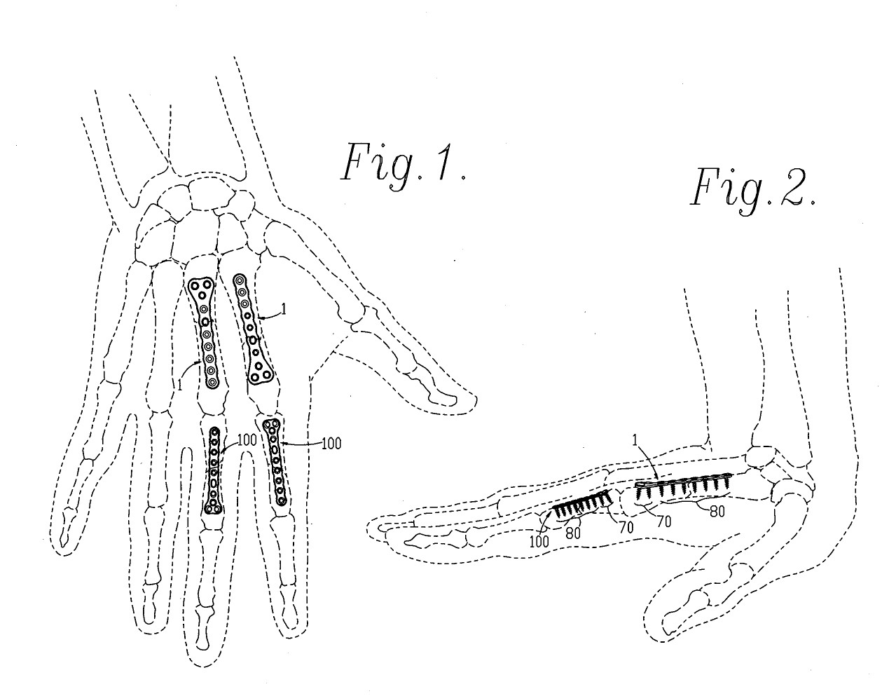

Referring to FIGS. 1 and 2, the present general inventive concept provides a fracture fixation fixed-angled locking plate 1 made of stainless steel or titanium alloy. The plate 1 is placed against and fixed to a shaft or diaphysis of a bone such as tubular bones of the hand including, but not limited to the bones of the fingers, e.g., metacarpal bones and/or phalanges.

The plate 1 has a body 10 that extends along a longitudinal axis. The plate 1 has a first portion 30 of the body 10 and a second portion 60 of the body 10 on either end of the plate 1, as illustrated in FIG. 3. The body 10 has two sidewalls 12 and 14 that extend the longitudinal axis of the body 10. The body 10 also has an upper surface 16 and a lower bone-engaging surface 18 that extend the longitudinal axis of the body 10.

As the sidewalls 12 and 14 extend along the second portion 60, the sidewalls 12 and 14 are parallel to each other or at least substantially parallel to each other. As the sidewalls 12 and 14 extend along the first portion 30, the sidewalls 12 and 14 diverge from each other, i.e., flare out or extend outwardly to form a buttress 40. The sidewalls 12 and 14 begin to diverge at or adjacent to the point where the first portion 30 and second portion 30 meet.

As the upper and lower surfaces 16 and 18 extend along the second portion 60, the upper and lower surfaces 16 and 18 are parallel to each other or at least substantially parallel to each other. As the upper and lower surfaces 16 and 18 extend along the first portion 30, the upper surface 16 diverges away from the lower surface 18, the lower surface extending along both the first and second portions 30 and 60 in the same plane. The upper and lower surfaces 16 and 18 begin to diverge at or adjacent to the point where the first portion 30 and second portion 30 meet.

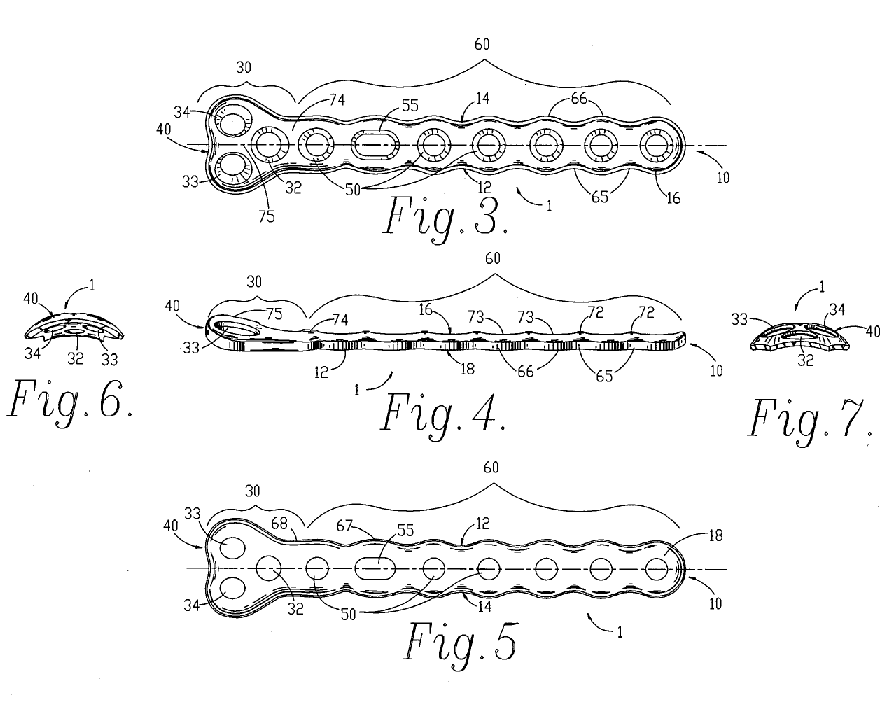

As illustrated in FIGS. 4, 6, and 7, the buttress 40 extends at an upward angle of approximately 12 degrees from the longitudinal axis of the body 10, and outward via both sidewalls 12 and 14 with respect to the second portion 60, the second portion 60 having a substantially uniform shape. The buttress 40 has a thickness that is thinner than the other part of the body 10 and may taper to a lesser thickness moving away from the second portion 60, as illustrated in FIG. 6. The buttress 40 at least partially extends onto or fits over a metaphysis of a bone such that the metaphysis is partially engulfed by the buttress 40. In this manner, the plate 1 is anchored against the metaphysis causing the plate 1 to be better secured to the bone.

The plate 1 has a plurality of apertures including a first set of apertures 32, 33, and 34 and a second set of apertures 50 to provide a variety of mounting orientations for the plate 1, thereby providing increased mounting versatility of the plate 1. The apertures 32, 33, 34, and 50 are conical or tapered inwardly toward the bone-engaging surface 18 of the plate 1. The screws to be accommodated by the apertures 32, 33, 34, and 50 may have a screw head with a tapered shape that substantially corresponds to and matches the tapered shape of the apertures 32, 33, 34, and 50.

The first set of apertures 32, 33, and 34 extend through the first portion 30, as illustrated in FIG. 3. The exemplary embodiment includes three apertures 32, 33, and 34. It is foreseen, however, that only two or one apertures may be employed in the first portion 30.

Aperture 32 is centered on the longitudinal axis of the body 10. Apertures 33 and 34 are centered on an axis that is perpendicular to the longitudinal axis of the body 10, and are offset on either side of aperture 32. The conical shape of the apertures 32, 33, and 34 vary to enable the apertures 32, 33, and 34 to receive and direct set screws 70 at different angles, such as angles of 10-45 degrees and preferably 15-45 degrees, relative to a plane defined by the upper surface 16.

The pegs or set screws 70 are preferably angled by the apertures 32, 33, and 34 in more than one direction, e.g., in two dimensions, to reflect the bone size increase from the diaphysis to the metaphysis. This permits penetration by the set screws 70 into an area of the bone that is more expansive than an area covered by the plate 1, i.e., into an area greater than a width of the plate 1 and/or beyond an edge 12 or 14 of the plate 1, as depicted in FIG. 2.

The second set of apertures 50 extend through and along the second portion 60, as illustrated in FIG. 3. The exemplary embodiment includes six apertures 50. It is foreseen, however, that more or fewer apertures may be employed in the second portion 60.

The apertures 50 extend in a line along the longitudinal axis of the body 10 and are of equal size and shape such that pegs or set screws 80 inserted therein are directed into the bone at angles that are equal to each other. While the majority of apertures 50 are equidistant from each other, one of the apertures 50 is separated from the other apertures 50 by an oblong aperture 55 to provide tolerance for movement of the plate 1 after initial placement of the plate 1 on the bone.

The oblong aperture 55 is designed to accept a first of the set screws 80 to affix the plate 1 to the bone such that as the first of the set screws 80 penetrates the bone, the plate 1 becomes rigidly clamped to the bone. Specifically, the first of the set screws 80 is initially installed through an approximate center of the oblong aperture 55 to secure the plate 1 to the shaft of the bone. If it is determined that the plate 1 is not in an ideal position and repositioning of the plate 1 is desired, the first of the set screws 80 can be loosened from the oblong aperture 55 to permit the plate 1 to be slid along a length of the oblong aperture 55 with respect to the first of the set screws 80 and without completely removing the first of the set screws 80 from the bone. If the first of the set screws 80 is initially installed near the center of the oblong aperture 55, the plate 1 may be slid in either direction relative to the first of the set screws 80 with respect to the oblong aperture 55.

When the plate 1 is in an ideal position, one or all of the set screws 70 are then installed through the fixed-angled locking apertures 32, 33, and 34, which direct the set screws 70 into the metaphysis of the bone. Any of the apertures 32, 33, and 34 can initially be used to hold the fracture reduced (by inserting a guide wire through it) while the other aperture(s) are drilled and filled with the appropriate length screw to stabilize the fracture. After the set screws 70 are installed, the set screws 80 are installed through the fixed-angled locking apertures 50, which direct the set screws 80 into the shaft of the bone.

The sidewalls 12 and 14 are waved running along a substantially S-shaped path extending inward and outward opposite to each other along the longitudinal axis of the body 10. The sidewalls 12 and 14 have inwardly-extending portions 65 that extend inwardly when the sidewalls 12 and 14 are adjacent to areas of the second portion 60 between the apertures 50. The sidewalls 12 and 14 have outwardly-extending portions 66 that extend outwardly when the sidewalls 12 and 14 are adjacent to apertures 50. The inwardly-extending portions 65 and outwardly-extending portions 66 extend inward and outward along the longitudinal axis at the same distance along the entire length of the second portion 60. The lengths of the outwardly-extending portions 66 increase as the sidewalls 12 and 14 extend from the second portion 60 to the first portion 30 along the longitudinal axis of the body 10.

For instance, the outwardly-extending portions 67 adjacent to the oblong aperture 55 is longer along the longitudinal axis of the body 10 relative to the outwardly-extending portions 66 on a side of the oblong aperture 55 opposite to the first portion 30. Likewise, the outwardly-extending portion 68 adjacent to the meeting point of the first portion 30 and the second portion 60 is longer than the outwardly-extending portion 67. The inwardly-extending portions 65 and outwardly-extending portions 66 permit the plate 1 to be as thin as possible while maintaining structural integrity.

The upper wall 16 is waved running along a substantially S-shaped path extending inward and outward along the longitudinal axis of the body 10. The upper wall 16 has inwardly-extending portions 73 that extend inwardly toward the lower bone-engaging surface 18 when the upper wall 16 is adjacent to apertures 50. The upper wall 16 has outwardly-extending portions or peaks 72 that extend outwardly when the upper wall 16 is adjacent to areas of the second portion 60 between the apertures 50. The inwardly-extending portions 73 and outwardly-extending portions 72 extend inward and outward along the longitudinal axis at the same distance along the entire length of the second portion 60. The lengths of the outwardly-extending portions 72 increase as the upper wall 16 extends from the second portion 60 to the first portion 30 along the longitudinal axis of the body 10.

For instance, at the meeting point between the first portion 30 and second portion 60, an outwardly-extending portion 74 is formed that has a length greater than the outwardly-extending portions 72 along the longitudinal axis of the body 10 on a side of the oblong aperture 55 opposite to the first portion 30. Likewise, the outwardly-extending portion 75 between the apertures 34 and 35 has a length greater than the outwardly-extending portion 74. The inwardly-extending portions 73 and outwardly-extending portions 72 permit the plate 1 to be as thin as possible while maintaining structural integrity.

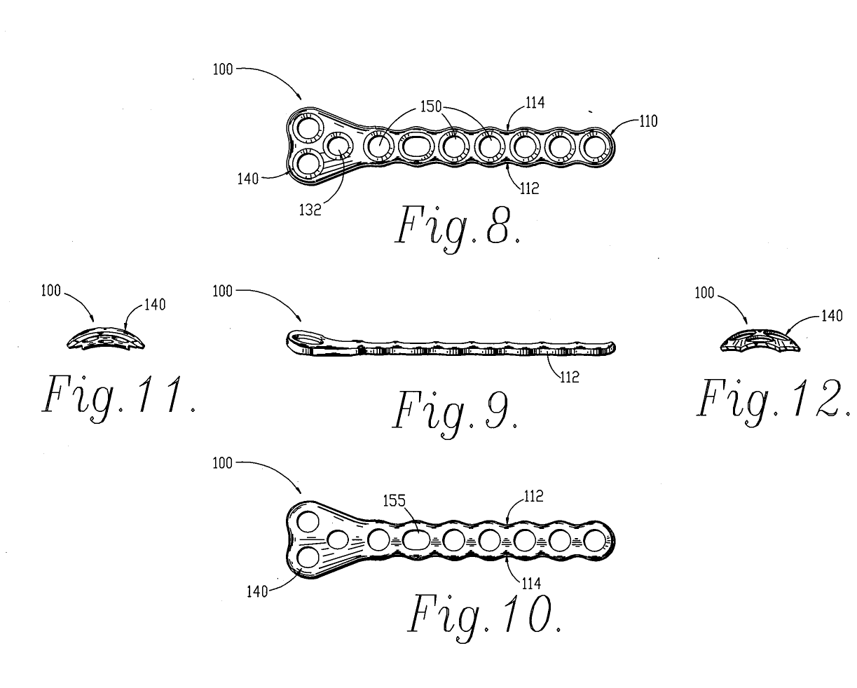

The plate 1 may have a thickness of 2.0 mm and 2.4 mm. FIGS. 3-7 illustrate plate 1, which is 2.4 mm. FIGS. 8-12 illustrate a plate 100 that is substantially identical to plate 1 except it is 2.0 mm. Plate 100 accommodates the set screws 70 and 80 of plate 1. It is foreseen, however, that other plates may be made smaller, e.g., with a thickness of 1.4 mm and 1.6 mm, and still accommodate set screws 70 and 80 of plate 1 or may be made smaller and only accommodate set screws or pegs that are smaller than set screws 70 and 80.

To accommodate the set screws 70 and 80 of plate 1 in relatively smaller plate 100, sidewalls 112 and 114 of body 110 are closer to apertures 150, as illustrated in FIG. 8. To maintain sufficient structural integrity of the plate 100, there is a smaller angle of expansion of sidewalls 112 and 114 as the body 110 forms a buttress 140. The smaller angle of expansion results in a thicker neck portion of the buttress 140 between aperture 132 and an adjacent aperture of apertures 150.

It is foreseen that the plate 1 may be placed such that the buttress 40 is adjacent to an upper or lower condyle of a metacarpal. Likewise, it is foreseen that the plate 100 may be placed such that the buttress 140 is adjacent to an upper or lower condyle of a phalanx. In this manner, the plates 1 and/or 100 may be placed in either a forward or backward direction, as illustrated in FIGS. 1 and 2.

The set screws 70 and 80 may be locking and/or non-locking and may have a screw head and/or be headless, locking and/or non-locking, fully threaded and/or lag (partially threaded) and/or cannulated. The 2.0 mm and 2.4 mm screws may be cannulated with a passage through an interior length thereof to accommodate a pre-inserted guide wire or the like.

While the present general inventive concept is intended for tubular bones of the hand, it is foreseen that the present general inventive concept may be employed to stabilize fractures of small bones other than tubular hand bones, such as but not limited to the tip of the elbow or the lateral side of the ankle.

Although a few embodiments of the present general inventive concept have been illustrated and described, it will be appreciated by those skilled in the art that changes may be made in these exemplary embodiments without departing from the principles and spirit of the general inventive concept, the scope of which is defined in the appended claims and their equivalents.

What is claimed is:

1. A tubular hand bone fracture fixation apparatus for use with a plurality of screws, the apparatus comprising:
an elongated body defining a longitudinal axis, the elongated body having a first portion and a second portion with an upper surface and a bone-engagement lower surface;
a first set of apertures through the first portion to orient a number of the plurality of fixation screws along two or more axes;
a second set of apertures through the second portion to orient a number of the plurality of fixation screws along a common axis; and
an oblong aperture located within the second set of apertures to orient a set screw,
wherein the first portion includes a support-enhancement element.

2. The apparatus according to claim 1, further comprising:
a plurality of peaks along the upper surface between the second set of apertures. wherein a length of the plurality of peaks successively increases from the second portion to the first portion.

3. The apparatus according to claim 1, further comprising:
sidewalls of the elongated body extending along the longitudinal axis,
wherein the sidewalls extend substantially parallel to each other along the second portion of the elongated body, and the sidewalls diverge along the first portion of the elongated body.

4. The apparatus according to claim 3, further comprising:
a plurality of inward portions of the sidewalls that narrow the elongated body; and
a plurality of outward portions of the sidewalls that widen the elongated body.

5. The apparatus according to claim 4, wherein each of the plurality of inward portions are adjacent to a non-aperture area of the elongated body, and each of the plurality of outward portions are adjacent to one of the second set of apertures.

6. The apparatus according to claim 4, wherein a length of the plurality of outward portions successively increases from the second portion to the first portion.

7. The apparatus according to claim 1, wherein the first set of apertures are conical tapered inward and angled with respect to a central axis of each of the apertures.

8. The apparatus according to claim 1, wherein the support-enhancement element has an interior with an upper wall and three walls that depend from the upper wall.

9. The apparatus according to claim 1, wherein the support-enhancement element is formed by parallel side surfaces of the elongated body that diverge outward from each other.

10. The apparatus according to claim 1, wherein the support-enhancement element is a buttress, a buttress cavity, or a buttress concavity.

11. The apparatus according to claim 1, wherein the bone-engagement lower surface flares upward and outward at the support-enhancement element to form a concavity that extends onto a metaphyse of a tubular bone.

12. The apparatus according to claim 1, wherein the screws include a fixation screw and a set screw.

13. The apparatus according to claim 1, wherein the upper surface and the bone-engagement lower surface of the elongated body extend substantially parallel to each other along the second portion, and the upper surface extends at an upward angle of approximately 13 degrees from the bone-engagement lower surface along the first portion.

14. The apparatus according to claim 1, further comprising:
an elongated aperture extending axially in a direction substantially aligned with the longitudinal axis.

15. The apparatus according to claim 1, wherein the elongated body tapers in thickness from the second portion to the first portion and is thinnest proximate to an edge of the support-enhancement element.

16. The apparatus according to claim 1, wherein each of the first set of apertures and the second set of apertures are conically tapered inward toward the bone-engagement lower surface of the elongated body.

17. The apparatus according to claim 1, wherein the apparatus is a metacarpal plate or phalanx plate and is reversible along a metacarpal or phalanx.

18. The apparatus according to claim 1, wherein the first set of apertures include a lower first aperture that is on the common axis of the second set of apertures, and upper second and third apertures that are opposite to each other on either side of the first aperture on another axis that is perpendicular to the common axis.

19. The apparatus according to claim 1, wherein the first set of apertures orient one or more screws at an angle between 10 and 45 degrees relative to the planar top surface and along a z-axis thereof.

20. The apparatus according to claim 1, wherein the elongated body has a thickness of 1.6 mm, 2.0 mm, or 2.4 mm.
